(19) United States
(12) Patent Application Publication
(10) Pub. No.: US 2006/0211951 A1
(43) Pub. Date: Sep. 21, 2006

(54) Implantable bladder sensor

(76) Inventors: Zoran Milijasevic, Bayview Heights (AU); Andrew Barriskill, Mosman (AU); Ian Bruinsma, Kings Langley (AU); Tom Niec, Mt. Pritchard (AU)

Correspondence Address: JAGTIANI + GUTTAG, 10363-A DEMOCRACY LANE, FAIRFAX, VA, 22030, US

(21) Appl. No.: 10/515,840

(22) PCT Filed: May 28, 2003
(86) PCT No.: PCT/AU03/00653

§ 371 Date: Sep. 14, 2005

(30) Foreign Application Priority Data
May 29, 2002 (AU) PS 2643

(51) Int. Cl. A61B 5/05

(52) U.S. Cl. 600/547

(57) ABSTRACT

An implantable sensor for monitoring the condition of an organ, such as the bladder, of a recipients' body. The sensor (100) comprises a carrier (102), such as a tubular housing that is attachable to the outer surface of the organ. An impedance-varying means (104), such as a saline solution, is carried by the carrier (102). The impedance-varying means (104) within the carrier varies as the condition of the organ changes to cause in output state of the sensor (100). The sensor (1000) can be used for the purposes of monitoring the filling of the bladder, making bladder volume information to the recipient or another person, and causing an alarm signal to be generated when a preset bladder volume is reached.

### FIELD OF THE INVENTION

This invention relates to sensing a parameter of an organ of a person's body. More particularly, the invention relates to a device for sensing a parameter of a visceral organ of a person's body.

### BACKGROUND TO THE INVENTION

Urinary and faecal incontinence are major health problems, particularly with an ageing population, for which there is no well-accepted medical treatment. Such conditions can affect men and women of all ages and is a particularly prevalent problem for individuals suffering from spinal cord injury, such as tetraplegics, who may be unable to exercise volitional control over the detrusor muscle of his or her bladder and other functions such as bowel evacuation.

With the advent of Functional Electrical Stimulation (FES) Systems, such as that developed by the Applicant and described in International Patent Application No. PCT/AU03/00044, the contents of which are incorporated herein by reference, it is possible to control bladder function and, more particularly, incontinence and evacuation of a bladder and other organs innervated via the sacral region of a person's spinal cord.

To enable this control to be effected, at least one parameter of the organ being controlled needs to be monitored. This is particularly important when the organ to be controlled is the bladder which has a capacity to fill and store urine. The ability to easily and effectively monitor the capacity of such an organ is therefore important to ensure that appropriate evacuation of the organ occurs that is dependent upon the state of the organ.

In this regard, it is a preferred feature of the present invention to provide a means that assists in monitoring parameters associated with the visceral organs of a body.

### SUMMARY OF THE INVENTION

According to one aspect, the present invention is an implantable sensor for monitoring the condition of an organ of a recipient's body including:

a carrier for attachment to an outer surface of the organ; and

an impedance-varying means carried by the carrier, the impedance of the impedance-varying means varying as the condition of the organ changes to cause a change in output state of the impedance-varying means.

The invention is intended particularly for use in monitoring the volume of a person's bladder. The carrier may be attached, for example, by being sutured, to an outer wall of the detrusor muscle of the bladder by means of an appropriate surgical procedure. As the volume of the bladder increases, the wall of the bladder stretches and changes shape.

The carrier may include an attachment means for attachment to an outer surface or wall of the organ, such as the bladder, so that a change in the shape of the outer surface or wall causes a change in the impedance of the impedance-varying means.

The sensor may be implemented in numerous ways. For example, in a first embodiment of the invention, the carrier may comprise a flexible tube, such as a silicone tube, having closed ends.

The impedance-varying means may comprise a conductive material or a conductive mixture containing a conductive material. In one embodiment, the conductive material may be a conductive fluid, such as a saline solution, contained in the tube. Distortion of the tube, for example, by stretching and/or displacement of the tube, may cause a change in impedance, for example, resistive impedance, of the conductive medium.

In this aspect, the impedance change may be enhanced by incorporating surface features on an inner wall of the tube that increase the influence of the change in the impedance of the conductive medium as a result of its being distorted.

In another embodiment of the invention, the impedance-varying means may include a displaceable element which is displacably arranged relative to the carrier, displacement of the element relative to the carrier causing the change in impedance. The change in impedance may be either a change in resistive impedance or inductive impedance. In other words, linear motion of the displaceable element relative to the carrier may cause a change in impedance which is monitored as a measure of the change in the condition of the organ, eg. the volume of the bladder.

In yet another embodiment of the invention, the carrier may be a container. The impedance-varying means may be arranged in the container, the impedance-varying means comprising a strip of non-conductive material on which a segmented conductive layer is superimposed thereon.

The strip may be divided into segments by transversely extending, spaced slits that cause the segments to separate as the change in the condition of the organ occurs, eg. as curvature of the detrusor muscle occurs due to increased volume of the bladder. Such a change in the curvature may cause adjacent segments of the conductive layer, on opposed sides of the slits in the strip, to move apart. As a result, a change, being an increase, in resistive output of the impedance-varying means may occur to provide an indication of increase in volume of the organ, such as the bladder.

In still a further embodiment of the invention, the carrier may be in the form of a sealed container in which the impedance-varying means is received.

The impedance-varying means may comprise a pair of overlying strips with conductors carried on facing surfaces of the strips, the change in condition of the organ, such as the bladder, resulting in relative displacement of the conductors on the strips to cause a change in impedance. The two strips may be anchored together at one end.

When used on the bladder and the volume of the bladder is low, the conductors of the strips may be aligned and have a low resistive output. As the volume of the bladder increases the strips may follow the curvature of the wall of the bladder. As a result, the conductors of the strips may move out of alignment, increasing the resistive output to provide an indication of increase in bladder volume.

In a further aspect, the present invention is an implantable sensor for monitoring the volume of fluid present in a recipient's bladder including:

a carrier for attachment to an outer surface of the bladder; and

an impedance-varying means carried by the carrier;

wherein changes in the outer surface of the bladder as it expands upon receiving fluid are detected as changes in the impedance of the impedance-varying means.

In this aspect, changes in the outer surface of the bladder preferably cause the carrier and the impedance-varying means to undergo distortion.

In one embodiment of this aspect, the carrier can be an enclosed flexible housing, with, for example, the impedance-varying means being contained within the enclosed flexible housing.

In this aspect, the impedance-varying means can comprise a conductive material and distortion of the flexible housing can cause a change in impedance of the conductive material. This impedance change can be enhanced by incorporating surface features on an inner wall of the housing which increase the influence of the change in the impedance of the conductive material as a result of its being distorted. The conductive material is preferably a conductive fluid, such as a saline solution.

In another embodiment of this aspect, the impedance-varying means can comprise a strip of non-conductive material on which a segmented conductive layer is superimposed. The strip can be divided into segments that separate as the change in the condition of the bladder occurs.

In yet another embodiment of this aspect, the impedance-varying means can comprise a pair of overlying strips with conductors carried on facing surfaces of the strips, the change in the outer surface of the bladder resulting in relative displacement of the conductors on the strips to cause a change in impedance. In this embodiment, the two strips can be anchored together at one end.

In a still further aspect, the present invention is an implantable sensor for detecting variations in shape of an organ of a recipient's body comprising:

an elongate flexible housing attachable to an organ of the body;

a conductive material contained within the housing;

at least one electrode for detecting variations in impedance of the sensor;

wherein as the body organ changes shape, the housing also undergoes a change in shape which is detected by the electrode as a variation in the overall impedance of the conductive material within the housing.

In this aspect, the housing is preferably stretched on an increase in volume of the organ thereby reducing the cross-sectional area of the housing and so increasing the impedance of the conductive material within the housing.

In one embodiment of this aspect, the housing can comprise a series of chambers in fluid communication with each other for receiving the conductive material, each chamber being defined at at least one end by diametrically opposed raised formations in the housing and wherein, on stretching of the housing, the opposed raised formations move relatively closer to each other thereby further increasing the impedance of the conductive material within the housing.

The conductive material of this aspect is preferably a conductive fluid, such as a saline solution.

In this aspect, the organ monitored by the sensor is preferably the bladder.

### BRIEF DESCRIPTION OF THE DRAWINGS

The invention is now described by way of example with reference to the accompanying drawings, in which:

FIG. 1 shows a schematic, sectional side view of an implantable bladder sensor, in accordance with a first embodiment of the invention, for monitoring the condition of a person's bladder;

FIG. 2a shows a detailed section of part of the sensor of FIG. 1 in a relaxed state;

FIG. 2b shows the detailed section of the sensor of FIG. 1 in a stretched state;

FIG. 3 shows a top view of a variation of the first embodiment of the sensor of FIG. 1;

FIG. 4 shows a schematic, sectional side view of an implantable bladder sensor, in accordance with a second embodiment of the invention, for monitoring the condition of a person's bladder;

FIG. 5 shows a side view of an implantable bladder sensor, in accordance with a third embodiment of the invention, for monitoring the condition of a person's bladder;

FIG. 6 shows a side view of the sensor of FIG. 5, after a change in the condition has occurred;

FIG. 7 shows, on an enlarged scale, part of the sensor of FIG. 6;

FIG. 8 shows a three dimensional, schematic view of an implantable bladder sensor, in accordance with a fourth embodiment of the invention, for monitoring the condition of a person's bladder;

FIG. 9 shows a schematic plan view of the sensor of FIG. 8 when the bladder is in a first state;

FIG. 10 shows a schematic plan view of the sensor of FIG. 8 when the bladder is in a second state; and

FIG. 11 shows a schematic, three-dimensional view of the sensor when the bladder is in its second state.

### DETAILED DESCRIPTION OF THE INVENTION

The urinary bladder can be considered as a large, flexible muscular balloon that stores urine produced by the body in the kidneys. As the bladder fills and stores urine, it expands and changes shape in a manner that is dependent upon the volume of fluid it stores. In this regard, the shape and size of the bladder can provide a direct indication of the amount of urine present in the bladder, which can be used as an indication of when the bladder should be emptied.

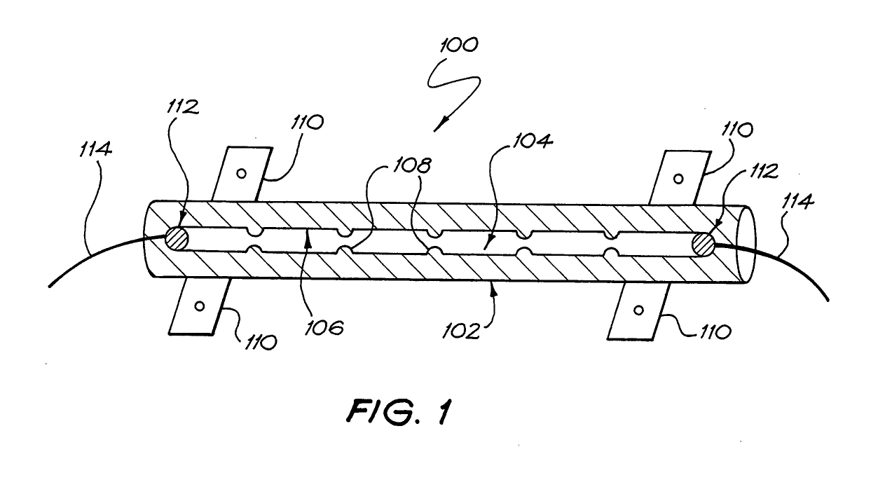

Referring initially to FIG. 1 of the drawings, an implantable bladder sensor, in accordance with a first embodiment of the invention, for monitoring the condition of a person's bladder is illustrated and is designated generally by the reference numeral 100. In particular, the sensor 100 is used for monitoring the volume of fluid present in a person's bladder by monitoring the change in size and/or shape of the bladder. While the person is normally a human, the technique can apply equally to other animals.

The sensor 100 includes a carrier 102 which carries an impedance-varying means 104. The carrier 102 is a tube having closed ends. The tube 102 contains the impedance-varying means 104 which is in the form of a conductive fluid, such as saline solution.

An inner wall 106 of the tube 102 carries an impedance enhancing arrangement in the form of a series of diametrically opposed raised formations 108, thereby forming a series of chambers for receiving the impedance-varying means 104. The role of these raised formations 108 and the chambers they form being discussed in more detail below.

The tube 102 has a plurality of pads 110 for securing the tube 102 to a wall of the bladder (not shown in this embodiment). Two electrodes 112 are positioned at opposite ends of an interior of the tube 102. The electrodes 112 are used to detect variations in the impedance of the saline solution 104. The electrodes 112 are connected via insulated wires 114 to a sensor/stimulator unit (not shown) which detects and measures changes in the impedance within the sensor 100.

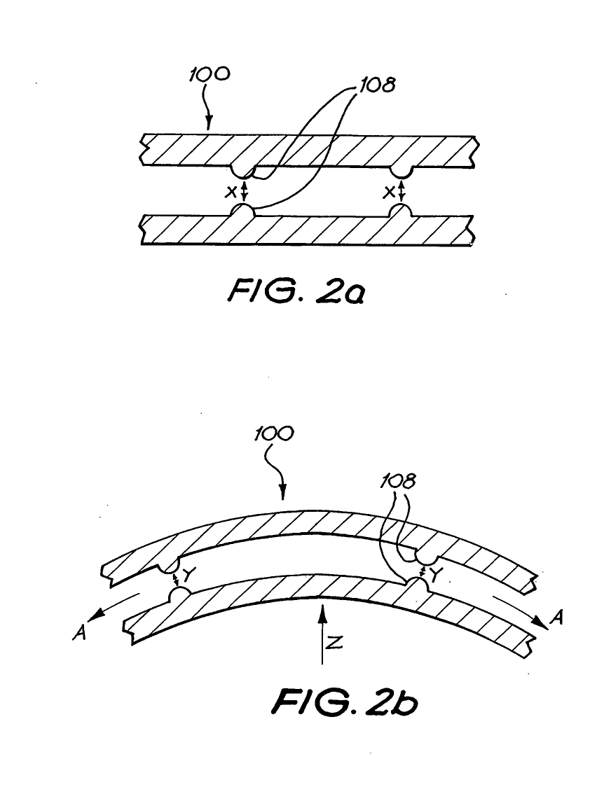

As can be seen more clearly in FIGS. 2a and 2b, the raised formations 108 on the inner wall 106 of the tube 102 serve to enhance (amplify) the effects of impedance change within the tube 102 of the sensor 100. The raised formations 108 form a series of open chambers along the tube 102, into which the conductive fluid flows. In use, when the sensor 100 is preferably sutured to the detrusor muscle of the bladder, as the bladder fills with fluid and changes shape and size, the detrusor muscle flexes and stretches to cause the tube 102 also to flex in a direction shown by arrow Z in FIG. 2b. In a relaxed state, as is shown in FIG. 2a, the conductive fluid (saline solution) 104 within the tube 102 is not overly impeded, and there is a definite conduction path between neighbouring fluid filled chambers within the tube 102 for conduction to occur as shown by the distance X between opposed raised formations 108.

However, when the bladder begins to fill and the wall of the bladder expands and flexes, the tube 102 of the sensor 100 is also stretched in the direction of arrow A. This causes the diameter of the tube 102 to reduce. As a result, the opposed raised formations 108 on the inner wall of the tube 102 are drawn closer together thereby reducing the cross-sectional area of the tube 102 as shown by dimension Y in FIG. 2b of the drawings. A restriction in the flow path between neighbouring chambers of the conductive fluid (saline solution) 104 results, which causes an increase in impedance of the sensor 100. The change in impedance of the sensor 100 in response to the change in size or shape of the bladder can be calibrated to provide an indication of the volume of fluid/urine present in the bladder. Whilst the presence of the raised formations 108 enhances the effect of impedance change of the sensor 100, an impedance change may still be detected in the absence of such raised formations 108 or by arranging the raised formations in a different manner than that described above.

FIG. 3 shows a top view of a variation of the embodiment of the sensor 100 of FIG. 1. With reference to FIG. 1 like reference numerals refer to like parts unless otherwise specified.

In this variation, the tube 102 is substantially hairpin or U-shaped. This allows the electrodes 112 to be arranged alongside each other which, in turn, allows the insulated wires 114 to be packaged together for convenience.

The sensor 100 is, once again, fixed to the wall of the bladder via the pads 110. Filling of the bladder causes the tube 102 to stretch in the direction of arrows B-B, thereby stretching the walls of the tube 102. The conductive medium 104 within the tube 102 (eg saline solution) experiences greater impedance in the stretched state of the tube 102 than in the relaxed state. As a result the change in impedance is calibrated to indicate the change in size or shape of the bladder representative of the amount of urine/fluid stored in the bladder. As described above, the impedance change could also be amplified by incorporating the raised formations 108 and creating chambers along the length of the tube 102 as discussed in relation to FIGS. 2a and 2b. It will be appreciated that, because the tube 102 has a length dimension that is much greater than its diameter, the effects of impedance change arising from any lateral expansion of the tube 102 due to flexing of the detrusor muscle of the bladder will be masked by the significantly greater increase in length of the tube 102.

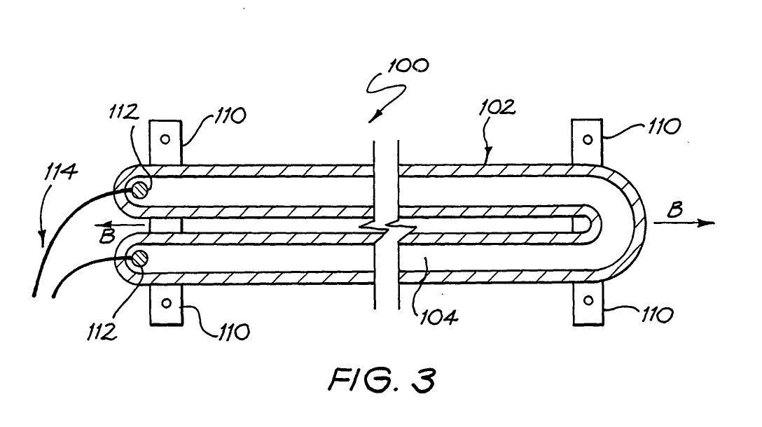

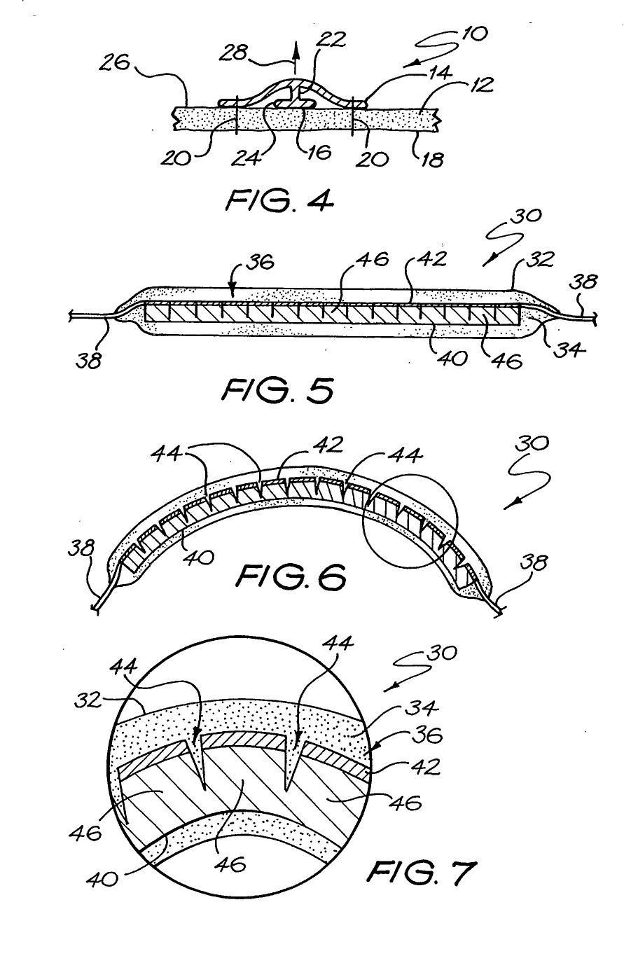

Referring to FIG. 4 of the drawings, an implantable bladder sensor, in accordance with a second embodiment of the invention, for monitoring the condition of a person's bladder is illustrated and is designated generally by the reference numeral 10. Once again, the sensor 10 is used in monitoring the volume of a person's bladder 12.

The sensor 10 includes a carrier 14 with an impedance-varying means 16 carried by the carrier 14.

In the embodiment of the invention shown in FIG. 4 of the drawings, the carrier 14 is a length of conductive silicone. The carrier 14 is attached to a wall 18 of the bladder 12 by, for example, sutures 20.

The impedance-varying means 16 is in the form of a probe 22 having a foot portion 24 which bears against an outer surface 26 of the wall 18 of the bladder 12.

When the bladder 12 has a low volume, the wall 18 of the bladder 12 is in a relaxed state and, as a result, the carrier 14 is similarly relaxed due to very little pressure, if any at all, being imparted thereto by the probe 22.

As the bladder 12 fills with liquid, the bladder changes size and/or shape and the wall 18 of the bladder 12 adopts a degree of curvature. This causes the probe 22 to be urged outwardly in the direction of arrow 28. Movement of the probe 22 in that direction stretches the length of conductive silicone constituting the carrier 14. This causes a change in resistance in the carrier 14. The change in resistance is indicative of the volume of liquid present in the bladder and as such can be calibrated to provide an indication of this volume to aid in determining when the bladder should be evacuated.

Referring to FIGS. 5 to 7 of the drawings, an implantable bladder sensor, in accordance with a third embodiment of the invention, for monitoring the condition of a person's bladder is illustrated and is designated generally by the reference numeral 30.

The sensor 30 includes a carrier in the form of a sealed bag 32. The sealed bag 32 contains a non-conductive material in the form of a non-conductive solution 34. An impedance-varying means 36 is contained within the bag 32 and has leads 38 projecting through sealed ends of the bag 32. The ends are sealed in a manner to inhibit escape of the solution 34 from the interior of the bag 32.

The impedance-varying means 36 comprises a strip 40 of a non-conductive material. A side of the strip 40 which, in use, constitutes an operatively outer side (ie. that side of the strip 40 which is furthest away from the wall of the bladder) carries a layer of a conductive material 42.

As illustrated more clearly in FIGS. 6 and 7 of the drawings, the strip 40 has transversely extending slits 44 defined therein to form segments 46. The layer 42 of conductive material can extend into the slits 44 as described below.

In use, the bag 32 is attached to the outer surface of a wall of the bladder by way of sutures or the like (not shown in this embodiment).

When the bladder is in a relaxed, empty state, the strip 40 lies in a substantially planar configuration so that the slits 44 are substantially closed. As a result, the conductive layer 42 is approximately continuous due to adjacent segments 46 abutting against each other providing a relatively short conducting path between the leads 38, and as such the sensor 30 has a first, low resistance state.

As the bladder fills and changes size and/or shape, the bag 32, together with the impedance-varying means 36, follows the curvature of the wall of the bladder as shown schematically in FIG. 6 of the drawings. As the strip 40 curves, the slits 44 open separating the segments 46. This causes an increase in the conductive path of the conductive layer 42, as the conductive layer extends into the slits 44, and a subsequent increase in the resistive output of the impedance-varying means 36. By monitoring the size and/or shape change of the bladder by monitoring the resistive output of the sensor 30, the volume of liquid present in the bladder can be readily determined to provide an indication of the filling state of the bladder.

Referring now to FIGS. 8 to 11 of the drawings, a fourth embodiment of an implantable bladder sensor for monitoring the condition of a person's bladder is illustrated and is designated generally by the reference numeral 50.

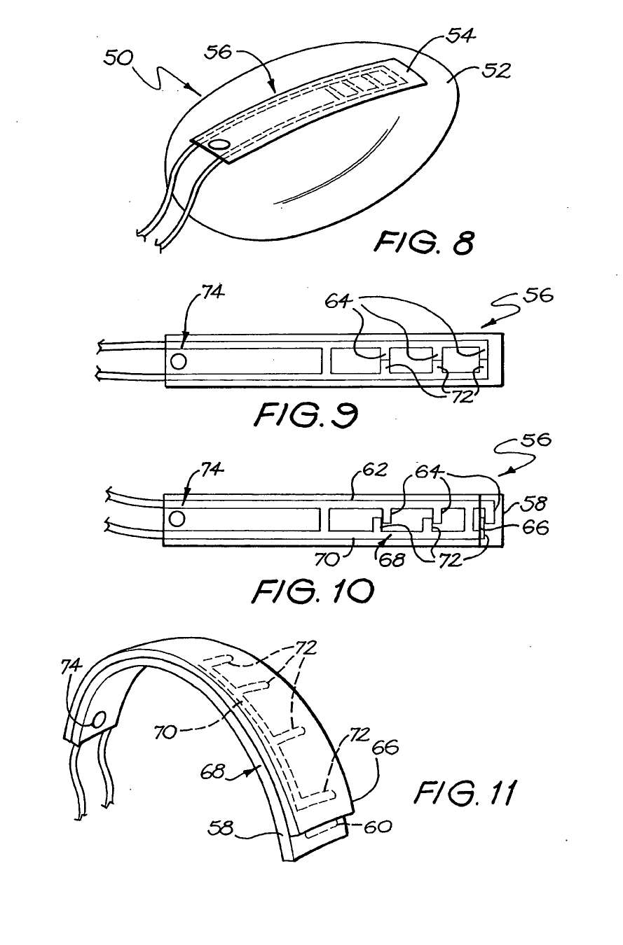

The sensor 50 includes a sealed bag or pouch 54 which is attached to an outer wall of a bladder 52 of the person's body. The pouch 54 carries an impedance-varying means 56 therein. The impedance-varying means 56 of the sensor 50 is shown in greater detail in FIGS. 9 to 11 of the drawings.

The impedance-varying means 56 comprises a first strip 58 of material on which a first set of conductors 60 is mounted. The arrangement of the set of conductors 60 is similar to that of a ladder which has been cut longitudinally. Thus, the set of conductors 60 includes a longitudinally extending conductor 62 with a plurality of longitudinally spaced, transversely extending portions 64. The set of conductors 60 is arranged on an operatively outer surface of the strip 58.

The impedance-varying means 56 comprises a second strip 66 which overlies the first strip 58. The second strip 66 carries a second set of conductors 68 on its surface in contact with the outer surface of the strip 58. The set of conductors 68 is the mirror image of the set of conductors 60 and comprises a longitudinally extending conductor 70 and a plurality of transversely extending portions 72.

The strip 58 and 66 are anchored together at one end as illustrated at 74.

In use, when the bladder 52 is in a relaxed, empty state, the wall of the bladder is substantially flat. As a result, the transverse portions 64 of the set of conductors 60 lie in register with the transverse portions 72 of the set of conductors 68, as shown in FIG. 9 of the drawings. As a result, the impedance-varying means 56 has a low resistive output.

As the bladder 52 fills and changes size and/or shape, its wall stretches and curves. This results the strips 58 and 66 adopting a curved configuration, as shown in an exaggerated state in FIG. 11 of the drawings, which causes displacement of the sets of conductors 60 and 68 relative to each other as shown in FIG. 10 of the drawings. Due to the transverse portions 64 of the set of conductors 60 now being out of alignment with the transverse portions 72 of the set of conductors 68, the resistive output of the impedance-varying means 56 increases. By monitoring the size and/or shape change of the bladder by monitoring the resistive output of the sensor 50, the volume of liquid present in the bladder can be readily determined to provide an indication of the filling state of the bladder.

As in the case of the first embodiment, the sensor of the second to fourth embodiments can also be used for monitoring reflex contractions of the bladder wall which can then be used by a stimulator to inhibit continuance of such contractions to improve continence of the person.

Accordingly, in the case of all four embodiments, the sensor is used for the purposes of:

(a) monitoring the filling of the bladder, making bladder volume information available to the person, and causing an alarm signal to be generated when a preset bladder volume is reached; and

(b) monitoring any early or premature (low volume) reflex contractions of the bladder and using detection of this condition to enable electrical neuromodulation, thereby inhibiting the reflex contractions and facilitating continence.

Hence, it is an advantage of the invention, that a sensor is provided which facilitates monitoring of a bladder's volume and reflex contractions of the bladder which can be used in a closed-loop stimulating system for enabling control of the bladder to be effected in persons who have lost the ability to control functioning of the bladder.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An implantable sensor for monitoring the condition of an organ of a recipient's body, the sensor including:
a carrier for attachment to an outer surface of the organ; and
an impedance-varying means carried by the carrier, the impedance of the impedance-varying means varying as the condition of the organ changes to cause a change in output state of the impedance-varying means.

2. The sensor of claim 1 in which the carrier includes an attachment means for attachment to an outer surface of the organ so that a change in shape of the outer surface causes a change in the impedance of the impedance-varying means.

3. The sensor of claim 1 in which the carrier comprises a flexible tube having closed ends.

4. The sensor of claim 3 in which the impedance-varying means comprises a conductive material contained in the tube.

5. The sensor of claim 4 in which distortion of the tube causes a change in impedance of the conductive material.

6. The sensor of claim 5 in which the impedance change is enhanced by incorporating surface features on an inner wall of the tube which increase the influence of the change in the impedance of the conductive material as a result of its being distorted.

7. The sensor of claim 1 in which the impedance-varying means includes a displaceable element which is displacably arranged relative to the carrier, displacement of the element relative to the carrier causing the change in impedance.

8. The sensor of claim 7 in which linear motion of the displaceable element relative to the carrier causes a change in impedance which is monitored as a measure of the change in the condition of the organ.

9. The sensor of claim 1 in which the carrier is a container.

10. The sensor of claim 9 in which the impedance-varying means is arranged in the container, the impedance-varying means comprising a strip of non-conductive material on which a segmented conductive layer is superimposed.

11. The sensor of claim 10 in which the strip is divided into segments that separate as the change in the condition of the organ occurs.

12. The sensor of claim 1 in which the carrier is in the form of a sealed container in which the impedance-varying means is received.

13. The sensor of claim 12 in which the impedance-varying means comprises a pair of overlying strips with conductors carried on facing surfaces of the strips, the change in condition of the organ resulting in relative displacement of the conductors on the strips to cause a change in impedance.

14. The sensor of claim 13 in which the two strips are anchored together at one end.

15. The sensor of claim 1 wherein the organ is a bladder.

16. An implantable sensor for monitoring the volume of fluid present in a recipient's bladder, the sensor including:
a carrier for attachment to an outer surface of the bladder; and
an impedance-varying means carried by the carrier;
wherein changes in the outer surface of the bladder as it expands upon receiving fluid are detected as changes in the impedance of the impedance-varying means.

17. The sensor of claim 16 wherein changes in the outer surface of the bladder cause the carrier and the impedance-varying means to undergo distortion.

18. The sensor of claim 17 wherein the carrier is an enclosed flexible housing.

19. The sensor of claim 18 wherein the impedance-varying means is contained within the enclosed flexible housing.

20. The sensor of claim 16 wherein the impedance-varying means comprises a conductive material.

21. The sensor of claim 20 wherein distortion of the flexible housing causes a change in impedance of the conductive material.

22. The sensor of claim 21 wherein the impedance change is enhanced by incorporating surface features on an inner wall of the housing which increase the influence of the change in the impedance of the conductive material as a result of its being distorted.

23. The sensor of claim 22 wherein the conductive material is a conductive fluid.

24. The sensor of claim 21 wherein the impedance-varying means comprising a strip of non-conductive material on which a segmented conductive layer is superimposed.

25. The sensor of claim 24 in which the strip is divided into segments that separate as the change in the condition of the bladder occurs.

26. The sensor of claim 21 in which the impedance-varying means comprises a pair of overlying strips with conductors carried on facing surfaces of the strips, the change in the outer surface of the bladder resulting in relative displacement of the conductors on the strips to cause a change in impedance.

27. The sensor of claim 26 in which the two strips are anchored together at one end.

28. An implantable sensor for detecting variations in shape of an organ of a recipient's body, the sensor comprising:
an elongate flexible housing attachable to an organ of the body;
a conductive material contained within the housing;
at least one electrode for detecting variations in impedance of the sensor;
wherein as the body organ changes shape, the housing also undergoes a change in shape which is detected by the electrode as a variation in the overall impedance of the conductive material within the housing.

29. The sensor of claim 28 wherein the housing is stretched on an increase in volume of the organ thereby reducing the cross-sectional area of the housing and so increasing the impedance of the conductive material within the housing.

30. The sensor of claim 29 wherein the housing comprises a series of chambers in fluid communication with each other for receiving the conductive material, each chamber being defined at at least one end by diametrically opposed raised formations in the housing and wherein, on stretching of the housing, the opposed raised formations move relatively closer to each other thereby further increasing the impedance of the conductive material within the housing.

31. The sensor of claim 29 wherein the conductive material is a saline solution.

32. The sensor of claim 28 wherein the organ is a bladder.
